(19) United States
(12) Patent Application Publication
(10) Pub. No.: US 2019/0367449 A1
(43) Pub. Date: Dec. 5, 2019

(54) PHENYLCREATINE, ITS USE AND METHOD FOR ITS PRODUCTION

(71) Applicants: Ilya Vladimirovich DUKHOVLINOV, St. Petersburg (RU); Aleksei Viktorovich ALEKSEEV, St.Petersburg (RU)

(72) Inventor: Ilya Vladimirovich DUKHOVLINOV, St. Petersburg (RU)

(21) Appl. No.: 16/478,038

(22) PCT Filed: Jan. 18, 2018
(86) PCT No.: PCT/RU2018/050004

§ 371 Date: Jul. 15, 2019

(30) Foreign Application Priority Data
Jan. 20, 2017 (EA) 201700089

(51) Int. Cl. C07C 279/14; C07C 277/02; A61P 9/06; A61P 25/28

(52) U.S. Cl. CPC C07C 279/14; A61P 25/28; A61P 9/06; C07C 277/02

(57) ABSTRACT

The invention relates to the field of pharmaceutical chemistry, namely to new biologically active substances and their use and to a method of production. In particular, the invention relates to a derivative of creatine—phenylcreatine, its use as a functional analogue of creatine, as well as a nootropic agent and for the prevention or treatment of arrhythmia and a method of its production.

The invention relates to pharmaceutical chemistry, namely to new biologically active substances and their use and to a method of production. In particular, the invention relates to a derivative of creatine—a substance of general formula: NH═C(NH₂)—N(C₆H₅)—CH₂—COOH (C₁₀—H₁₃—N₃—O₂), N-benzyl-N-carbamimidoylglycine (hereinafter—phenylcreatine).

Creatine, or 2-(methylguanine)-ethane acid is a nitrogen-containing carboxylic acid, which is present in various mammalian tissues, namely, liver, kidneys, muscle, brain tissue, blood, and even is found in photoreceptor cells of the retina, spermatozoa and sensory hair cells of the inner ear (Wallimann T, Tokarska-Schlattner M, Schlattner U., The creatine kinase system and pleiotropic effects of creatine, Amino Acids. 2011 May; 40(5):1271-96. doi: 10.1007/s00726-011-0877-3).

Approximately 95% of the total pool of creatine is stored in skeletal muscle tissues. At a time when energy demand increases, in the mitochondria creatine reversibly reacts with adenosine triphosphate (ATP) to form ADP and creatine phosphate with a help of the enzyme creatine kinase. Creatine phosphate is a reserve of macroergic phosphate. However, in contrast to ATP hydrolysed by pyrophosphate bond O—P, creatine phosphate is hydrolyzed by phosphamide bond N—P, which leads to much greater energy effect of the reaction. Therefore, this reaction helps to maintain a constant pool of ATP at the time of its intense consumption. Other methods, such as glycolysis and oxidative phosphorylation, also replenish the stock of ATP, but much slower (Shulman, Rothman, Metabolism By In Vivo NMR, Wiley 2005). In skeletal muscles creatine phosphate concentration may reach 20-35 mM or more.

Thus, creatine has an ability to increase muscle reserves of creatine phosphate, potentially increasing the muscle's ability to resynthesis of ATP from ADP to replenish energy, which in turn promotes improvement in the muscles capacity and the muscle mass increase (WO 2010074591 A1). Accordingly, the known effects of creatine are the increase of muscles volume and strength, as well as the speed of their contraction. The increase in muscle volume and strength is partially due to the fact that more water is drawn into the muscle tissue, as a greater amount of creatine is stored in it, and creatine monohydrate binds water.

The heart expresses the enzyme creatine kinase to a greater extent than any other tissue in a mammalian body, and this promotes the efficiency of mitochondrial activity increase: the increase of cytoplasmic concentrations of phosphocreatine (not so much of the creatine itself) is associated with an increase in the efficiency of oxidative processes in mitochondria, probably due to the transfer of high energy phosphate groups. Phosphocreatine is known to be the main source of energy for cardiac tissue along with fatty acids, which are dominant during the normoxia periods (normal O₂ level) and phosphocreatine becomes increasingly important during periods of hypoxic stress. The whole system of creatine kinase plays an important role in the recovery of the heart during ischemic/hypoxic stress, as blocking the activity of creatine kinase impairs recovery, and the overexpression of creatine kinase contributes to it. After ischemia, increased activity of the transporter of creatine (without necessarily affecting creatine kinase), for greater inflow of creatine, is associated with improvement of postischemic contractility by about 30% (Lygate C A, et al. Moderate elevation of intracellular creatine by targeting the creatine transporter protects mice from acute myocardial infarction. Cardiovasc Res. (2012)). Increase of the activity of the creatine kinase system, as well as the influx of creatine into a cell, is considered as an advantage after cardiac injury (WO/EP97/06225, 1999).

Oral administration of creatine increases the creatine content in a body. Extensive research has shown that taking creatine in an amount of from 5 to 20 grams per day is effective in improving the working capacity and endurance of the muscles, increasing the maximal production force of muscles in men and women, especially when used as a supplement to a diet of athletes (WO5 94/02127, 1994). Creatine keeps the reserve muscle activity, reducing the metabolic acid level, which can cause muscle fatigue and burn-out.

Taking creatine reduces the need for its production in the body. After taking creatine monohydrate (“boot” phase and 19 weeks of intake), the number of predecessors of creatine is reduced to 50% (habituation) or up to 30% (acceptance), which implies a decrease in the level of endogenous synthesis of creatine. This is due to the properties of creatine and suppression of L-arginine: glycine amidinotransferase enzyme limiting the rate of synthesis of creatine, reduces it to 75% (McMorris T, et al. Creatine supplementation and cognitive performance in elderly individuals. Neuropsychol Dev Cogn B Aging Neuropsychol Cogn. 2007). This suppression may be beneficial to health, due to release the body of the function. The expected increase in homocysteine after intense exercise also decreases, and this is one of reasons why creatine is considered to be a cardioprotective supplement in the process of performing of heavy exercises.

Also creatine is recommended as a nutritional supplement for the elderly and vegetarians, due to the fact that in these people, a clear decrease in the content of creatine in muscles is noted (WO 97/45026), i.e. to compensate for the natural losses.

The subject of two recent studies was to elucidate the role that creatine plays in muscle recovery after workouts. In one of these experiments, 14 untrained subjects were randomly divided into two groups. Within five days prior to the training with weights and 14 days after it first group took creatine with carbohydrates, while the second—only carbohydrates. They performed the one leg presses, leg extensions and flexions of one leg in four sets often repetitions, the exercises being only eccentric (lowering the weight), with a 120-percent weight of the maximum in the concentric (lifting) movements. Eccentric contractions cause damage of a larger number of muscle fibers and more severe pain than concentric. To assess muscle damage, scientists tracked the release of two muscle enzymes.

The participants who took creatine in combination with carbohydrates, have achieved much better results than those who took only carbs. And if specifically, in the “creatine group” isometric muscle strength was greater by 21 percent, and isokinetic—by 10 percent.

Despite the fact that in this experiment the exact mechanism of the beneficial effect of creatine has not been studied, the authors of this study suggest that dietary supplementation increases buffering of calcium in the muscles, which in turn, lowers intracellular calcium and helps to contain muscle degradation. Creatine also speeds up protein synthesis in muscles and contributes to enhanced proliferation of stem cells, and this leads to the formation of new muscle fibers. All this, taken together, improves the recovery processes after the exercise (J Int Soc Sports Nutr. 6:13. 2009, J Sports Sci Med. 8:89-96. 2009).

Recent studies show that creatine promotes protein synthesis through stimulation of insulin-like growth factor 1 in muscles.

Creatine is used in the treatment of hyperglycemia and diabetes (U.S. Pat. No. 6,193,973, 2001). In healthy men, having a sedentary lifestyle, who used a loading protocol of creatine followed by 11-week maintenance period, the glucose response in glucose tolerance test is reduced by 11-22% (for 4-12 weeks, regardless of time), which was not associated with changes in insulin level or sensitivity (Rooney K B, et al. Creatine supplementation affects glucose homeostasis but not insulin secretion in humans. Ann Nutr Metab. 2003).

It is known that respiration of mitochondria is enhanced in skeletal muscles at a concentration of creatine 20 mM. The same thing happens in the cells of the hippocampus. It promotes endogenous PSD-95 clusters and subsequently the synaptic neurogenesis, which is considered secondary to the promotion of mitochondrial function. Mitochondrial function as such, apparently, promotes the growth and proliferation of neurons, and creatine, at least in vitro, plays an important role in this.

So, creatine, creatine phosphate and cyclocreatine (U.S. Pat. No. 6,706,764, 2004) are recommended for the treatment of diseases of the nervous system. For example, brain injuries tend to cause further damage of the cells, which is secondary to ATP depletion and creatine, apparently, maintains the permeability of mitochondrial membranes in response to brain damage which is believed to be related with its ability to preserve ATP. In rats and mice that received the injections of creatine (3 g/kg) for up to five days before craniocerebral trauma, using supplements managed to reduce the severity of craniocerebral trauma by 3-36% (depends on the time of application; admission for five days is associated with a higher efficiency than admission for one or three days), and dietary consumption of 1% creatine for four weeks has halved subsequent injuries. Daily consumption of creatine in rats, apparently, is able to halve the effects of brain injury. In children and adults with craniocerebral trauma (CCT), in six months of creatine admission in the amount of 400 mg/kg of body weight the following are reduced significantly—the frequency of headaches (from 93.8% to 11.1%), fatigue (from 82.4% to 11.1%), and dizziness (from 88.9 percent to 43.8%), compared to not blind control. Preliminary data show that headaches and dizziness associated with CCT can be eased with oral admission of creatine supplementations (Sullivan P G, et al. Dietary supplement creatine protects against traumatic brain injury. Ann Neurol. 2000).

There is a perception that endogenous creatine plays an important role in a number of cognitive functions, including learning, memory, attention, speech and language, and, perhaps, emotions (Allen P J, Creatine metabolism and psychiatric disorders: Does creatine supplementation have therapeutic value?, Neurosci Biobehav Rev. 2012 May; 36(5):1442-62. doi: 10.1016/j.neubiorev.2012.03.005).

However, the use of creatine and creatine phosphate decreases due to the decrease of solubility and instability in aqueous media at physiological pH rates (RU 2295261, 2007). It is also known that creatine is poorly absorbed from the gastrointestinal tract, so it often happens that orally creatine is taken in high doses, from about 5 g per 80 kg of body weight. This leads, primarily, to the increase in the cost of course of the drug, and it is also known that high doses of creatine can have a negative impact in the form of weight gain, gastro-intestinal disorders, inhibiting the synthesis of endogenous creatine, renal dysfunction or dehydration, to a lesser extent mood disorders and anxiety (Allen P J, Creatine metabolism and psychiatric disorders: Does creatine supplementation have therapeutic value?, Neurosci Biobehav Rev. 2012 May; 36(5):1442-62. doi: 10.1016/j.neubiorev.2012.03.005).

There are several ways to increase the bioavailability of creatine.

The intake of creatine monohydrate in the solution of simple carbohydrates increases the bioavailability of this supplement for muscles. Another method which enhances the effect of creatine monohydrate is its combination with substances that stimulate the secretion of the pancreatic hormone insulin. In several studies it has been shown that increasing the level of insulin in the blood results in a significant increase of creatine accumulation in the muscles. The majority of creatine transport systems works when stimulating organism for the production of insulin by a simple carbohydrate like dextrose. For this to wash down the drug not water is suggested, but a natural juice, especially a grape one, which is rich in carbohydrates.

So, to increase the availability of creatine for the muscles when administered orally (absorption through the stomach) the use of micronized creatine with sugar is known, the mechanism is through insulin (WO 2001/070238 A1), or with a simple carbohydrate, for example maltodextrin or dextrose, mechanism also using insulin (http://www.purenutrition.com.au/creatine-explained/), or creatine with dextrose, 18 g (http://www.livestrong.com/article/465112-how-much-dextrose-do-you-mix-with-creatine-for-bodybuilding/). An effective increase of strength and a change of body composition is known in men with the use of creatine together with glucose and also fenugreek (900 mg) when using 3.5 g of creatine (http://www.predatornutrition.com/articlesdetail?cid=fenugreek-improves-creatine-absorption-more-than-carbohydrates). Also a method of creatine delivery is known, in which this molecule is introduced in the matrix containing one or more sugar syrups; one or more modified starches; hydrocolloid component containing gelatin or a combination of gelatine and gellan; a solvent comprising glycerol, lower alkyl ester derivatives of glycerol, propylene glycol, polyalkylene glycol with a short chain, or a combination thereof; one or more sources of mono or divalent cations and one or more sources of water, in the delivery vehicle moisture content is from about 10% to about 30% by weight and a water activity is less than about 0.7 (US 2004/0013732 A1).

Despite the fact that the creatine in some quantities used to normalize the level of sugar in blood, such additional admission of “fast” carbohydrates causes over time insulin resistance and diabetes (Hjelmesæth, Jøran, et al. “Low serum creatinine is associated with type 2 diabetes in morbidly obese women and men: a cross-sectional study.” BMC endocrine disorders 10.1 (2010): 1.).

Currently, creatine is the main representative of the group of ergogenic components of sports nutrition and is available in different chemical forms (monohydrate, hydrotrate, alpha-ketoglutarate, tri—and dicreatine malate, citrate, ethyl ester of creatine, etc.). There is a large number of derivatives of creatine, such as, for example, pyruvate creatine (U.S. Pat. No. 6,166,249; RU2114823), derivatives of creatine and malonic, maleic, fumaric, orotic acids and taurine (CN 10/249338; U.S. Pat. Nos. 6,861,554; 6,166,249; CA 10/740263), esters of creatine, to increase the availability of creatine for muscles when administered orally (absorption through the stomach) (AU 2001/290939 B2), such as ethyl and benzyl (WO 02/22135), magnesium salt of creatine phosphate (CN 1709896) and others.

To improve absorbability and availability for tissues the use of salts of creatine is known (U.S. Pat. No. 7,479,560 B2). Compared to the creatine monohydrate, β-alaninate salt of creatine (the creatine β-alaninate salt) has a high solubility in organic solvents and aqueous solutions, in comparison to creatine and increased absorbability and bioavailability for tissues (WO 2011/019348 A1). It is also shown that a stable aqueous solution of the sulfate salt of creatine acid with a buffer agent and pH 7.5 used orally is faster absorbed by a body (WO 1999/043312 A1).

It is known that the monohydrate or pyruvate, or creatine ascorbate or α-ketoglutarates of creatine are easily absorbed and used to treat premenstrual syndrome in women (U.S. Pat. No. 6,503,951 B2). Dry creatine α-ketoglutarate, the molar ratio of 1:2, is used also to increase the period of storage at room temperature for up to a year (US20130184487).

The relative bioavailability of creatine hydrochloride is about 50% higher than of creatine monohydrate (U.S. Pat. No. 8,354,450 B2).

The disadvantage of these compounds is the lack of stability in the body and a low bioequivalence.

It is shown that creatine bicarbonate has an enhanced absorbability and bioavailability for tissues, compared with creatine (U.S. Pat. No. 8,466,198 B2). The use of creatine together with sodium bicarbonate allows to enhance interval swimming, but only at the beginning, and there are health risks because of the increased capture of sodium (http://kendevo.com/tag/creatine-absorption/).

It is shown that the absorption of creatine is increased with the use of α-lipoic acid (Effect of α-Lipoic Acid Combined With Creatine Monohydrate on Human Skeletal Muscle Creatine and Phosphagen Concentration. International Journal of Sport Nutrition and Exercise Metabolism, 2003, 13, 294-302), or propylene glycol, the absorbability—through the intestine (U.S. Pat. No. 5,773,473 A).

Use one safe molecule having the activity of creatine, wherein having a greater bioavailability and activity than creatine, and a high stability, is more preferable, respectively, the production of such derivative of creatine is an urgent task.

This problem is solved by a non-trivially proposed new molecule—phenylcreatine (N-benzyl-N-carbamimidoyl glycine).

The proposed molecule is new. The following analogues are known.

A combined use of creatine is known with esters of phenol for protection against the UVA and/or UVB rays, for the prevention and treatment of wrinkles, in the composition applied to the skin topically (AU 783758 B2), anti-aging (WO 2006/065920 A1). The combined use of creatine and oxybenzene (Oxybenzene) is known as a protection against the sun for the treatment of damaged skin, such a composition is applied to the skin (WO 2008/073332 A2, US2009098221 (A1)).

It is known to use topically a composition containing an oil and for antioxidant activity—creatine and polyphenol (US 2014/0315995 A1).

It is known to use creatine as a sweet taste improving organic acid additive, together with phenol as antioxidant, the composition also contains rebaudioside A and erythrite (RU2588540C2) or other substances (RU2472528C2).

It is known to use creatine in a composition with other components as an agent of cellular energy transport—a substance of aerobic energy metabolism of a cell, together with phenol as an antibacterial and antifungal agent (RU2288706C2), creatine for the regulation of pH, together with phenol as an antiseptic, antimicrobial or antibacterial agent, the composition is for the whitening of teeth (RU2505282C2).

A compound is known in which the creatine is bound with a ligand, wherein phenylalanine or phenyl serine can be the ligand, however the place of such connection is not specified (US 2011/0008306 A1). Prodrugs of creatine are known, i.e. compounds that decompose upon ingestion, where phenyl may be a substituent, however, in the document compounds of another structure than creatine are described, and the location of the phenolic group is not similar to the offered by the author of the present invention (WO 2016/106284 A2).

A compound is known on the basis of the creatine, an additional component is added on the NH group, the connection with the phenolic group is carried out without intermediate CH₂— bond, the phenolic group is connected with the heterocycle, one of the ring substituents—CH₂L, where L is an optional component (WO 2009/002913 A1).

A compound is known that is similar to phenylcreatine, however, the carboxyl group is replaced by another one (U.S. Ser. No. 09/127,233B2). Such structure provides another functionality.

Also phenylcreatine is known in which the linking of creatine with the phenolic group is via an amino group that also offers another functionality (WO 2015/120299 A1).

However, a molecule of creatine is considered phenylcreatine prototype, since creatine derivatives, including those described above, have a rather different functionality, due to the structure, as also compositions containing creatine and phenol.

The technical result from the use of the invention is to substantially decrease the dose of the substance applied and the frequency of its application to achieve the desired effect: 125 mg of phenylcreatine per 80 kg of weight, compared to 5 g of creatine and other forms of creatine per 80 kg of weight, to obtain the desired results associated with the muscle mass increase, muscular strength increase, improving performance (the ability to perform more sets/repeats), the weight gain.

The technical result from the use of the invention is in the acceleration of post-exercise recovery, with a substantial reduction of the dose of the applied substance—instead of 72 hours it happens within 24 hours.

The technical result from the use of the invention is the increase of the duration of the effect of the applied substance—it is maintained for 48 hours in the case of the proposed phenylcreatine, unlike creatine, which is only effective for 16 hours.

The technical result from the use of the invention is to maintain the effects in the absence of sleep.

In addition, the technical result from the use of the invention is in enhancing the effect of creatine even with low dosages of the proposed molecule, which is expressed, in particular, in the enhanced regeneration of nerve tissue and normalization of a blood supply of the brain.

The technical result is also expressed in expanding the range of derivatives of creatine, which will allow to achieve the desired result in case of absence of possibility of analogues use.

The author of the present invention also found that this compound has additional properties, in relation to the known for creatine.

Extrasystoles is the most frequent type of arrhythmia and is diagnosed in patients with the widest range of diseases, not only cardiac ones (http://www.lvrach.ru/2005/04/4532384/). For example, it is known that metabolic and carbohydrate metabolism disorders (diabetes, insulin resistance) lead to a violation of the restoration of ATP in the cell and lead to the formation of a persistent extrasystoles (Balashov, V. P., Balykov a L. A., Kostin I., Sernov L. N. Experimental and clinical pharmacology No. 2, 17-19 1996). However, the etiology of extrasystoles determines the choice of antiarrhythmic drugs only to some extent.

The main types of drugs for the treatment of arrhythmia: beta-blockers, inhibitors of production of angiotensin converting enzyme and drugs to completely eliminate the signs of arrhythmia, wherein their efficiency is not more than about 70% (http://www.aritmia.info/ekstrasistolija). However, the phenylcreatine does not act similarly to the these means, —its effect is not transient, as of antiarrhythmic agents, and its mechanism of action is not through blocking and inhibiting the respective molecules, but is probably due to restoration of the energy supply of cells, which allows for effectively and safely dealing with extrasystoles.

The technical result is expressed, firstly, in expanding the range of drugs for the prevention and treatment of extrasystoles, allowing at impossibility of use of analogs to achieve the desired result.

The technical result is also expressed in increasing the safety and efficiency of the prevention and treatment of cardiac extrasystoles, due to the implementation of a body-safe mechanism and use of molecules of the proposed structure, respectively.

Nootropics—means that have a specific positive impact on higher integrative functions of the brain. They improve mental activity, stimulate cognitive functions, learning and memory, increase brain resistance to various damaging factors, including extreme stress and hypoxia. In addition, nootropics have the ability to reduce neurological deficit and improve corticosubcortical connection. To designate substances of this group, there is a number of synonyms: neurodynamic, neuro-regulatory, neuroanabolic or eutotrophic agents, neurometabolic cerebroprotectors, neurometabolic stimulants. These terms reflect a common property of drugs—the ability to stimulate the metabolic processes in the nervous tissue, especially in various disorders (anoxia, ischemia, intoxications, injury etc.), returning them to a normal level (http://www.rlsnet.ru/fg_index_id_46.htm).

The technical result is also expressed in expansion of a spectrum of nootropic agents that will allow in case of impossibility of analogues use to achieve the desired result.

The technical result is also expressed in increasing safety and efficiency of the prevention and treatment of conditions and diseases that can be adjusted in one degree or another by nootropic agents, through the implementation of body-safe mechanism and use of molecules of the proposed structure, respectively.

Thus, the use of creatine and phenol, and molecules on their basis it is not known for obtaining the above-mentioned technical results.

All the above-mentioned technical results are achieved using the proposed phenylcreatine molecule.

Creatine is synthesized by the body from 3 amino acids: glycine, arginine and methionine. In humans the enzymes involved in the synthesis of creatine are localized in the liver, pancreas and kidneys. Neurons also possess the ability to synthesize creatine. The connection of two amino acids forms guaninoacetate, and after methylation of this molecule creatine is formed. Two enzymes participate in this process, one of them is a formed by ornithine, while the second is a used S-adenylmethion (methyl donor) (Braissant O, Henry H. AGAT, GAMT and SLC6A8 distribution in the central nervous system, in relation to creatine deficiency syndromes: A review. J Inherit Metab Dis., 2008) Creatine can be produced in any of these organs and then transported through the blood and absorbed by tissues requiring high energy consumption such as the brain and skeletal muscles, through an active transport system.

As the proposed by the author of the present invention phenylcreatine is a new molecule, method of its production is not known. Accordingly, the technical result from the use of the method is in obtaining phenylcreatine according to the invention, and quite simply.

### SUMMARY OF THE INVENTION

Phenylcreatine is given (N-benzyl-N-carbamimidoyl glycine, NH═C(NH₂)—N(C₆H₅)—CH₂—COOH (C₁₀—H₁₃—N₃—O₂),) of the following structure:

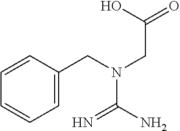 [SMILES: N=C(N)N(CC(=O)O)Cc1ccccc1]

Molecular formula: C10-H13-N3-O2; M=207, 299.

The substance is a friable white powder.

Phenylcreatine is synthesized by simple chemical transformation of urea (carbamide) and N-benzylglycine through the following reaction:

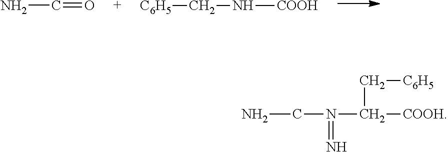

The reaction proceeds at temperature range from a room one to +65° C. for 24-96 hours at normal atmospheric pressure and normal humidity. The largest yield was observed when the reaction was carried out at a room temperature for 96 hours.

The proposed molecule can be used as a functional analogue of creatine, as well as a nootropic agent and for the prevention or treatment of extrasystoles.

Laboratory studies has been performed showing specific examples of implementation of the given invention. The obtained results of laboratory tests are illustrated by FIG. 1 and examples 1-5.

### BRIEF DESCRIPTION OF THE DRAWINGS

FIG. 1 Graphs of dynamics of the duration of mice run to complete exhaustion in the experiment described in example 3.

### EXAMPLE 1. PRODUCTION OF PHENYLCREATINE

N-benzylglycine weighing 429 mg, and 0.5 ml of distilled water were mixed in a round-bottom flask of 10 ml volume. Then 152 mg of NaCl were added to the mixture. Further, using a magnetic stirrer the mixture was stirred at room temperature for 10 minutes. In a small glass 206 mg of cyanamide and 0.2 ml of distilled water were added. Then a drop of solution of ammonia was added in catalytic quantities. The mixture was quickly mixed by gentle inverting, and then a mixture of cyanamide was added to a mixture of N-benzylglycine. The resulting mixture was stirred for one hour at room temperature. After 96 hours of incubation at room temperature and normal atmospheric pressure the product, namely phenylcreatine, N-benzyl-N-carbamimidoyl glycine, was precipitated. The crystals were transferred to a clean container with a volume of 10 ml.

Purification of the sample was performed by recrystallization with the use of 1-2 ml of boiling distilled water. Then the solution was cooled to until its temperature became a room one. Then the solution was cooled on an ice bath for five minutes and dried in vacuum.

The product was received also by incubation at higher temperatures up to 65° C., it was crystallized after from 24 hours to a week. If phenylcreatine remained in the solution, the solution was filtered until the dry crystals of the substance were discovered, vacuum filtration was used. The output of phenylcreatine ultimately amounted to 65-80%. Mass spectrum, found: m/z: MYR 207.2. Calculated: M 209.

### EXAMPLE 2. THE STUDY OF THE STABILITY OF PHENYLCREATINE COMPARED TO CREATINE IN AQUEOUS SOLUTION AND IN BLOOD

The study of the stability of phenylcreatine and creatine in aqueous solution and in human blood was carried out as follows.

For the preparation of solutions of test substances on an analytical balance the exactly weighed phenylcreatine and creatine were taken. The calculated amount of double-distilled water was added to them to obtain a concentration of 1 mg/ml. A part of the solution was diluted 10 times, and the sample was immediately analyzed. Further, that solution was kept at room temperature and after 3 hours the analysis was repeated.

Further according to the method described in Dunnett, Harris & Orme (1991) Reverse phase ion-pairing high performance liquid chromatography of phosphocreatine, creatine and creatinine in equine muscle. Scand. J. Clin. Lab. Invest. 51, 137-141, page 139, aliquot (c), creatinine, creatine and phosphocreatine were removed from the samples used for obtaining of the blood serum for mixing, to obtain more accurate results.

1 ml of water or prepared according to the method above blood serum was added to 200 μl of a solution in water of initial substance with a concentration of 2-3 mg/ml, shaken and a sample of 200 μl volume was immediately taken and initial concentration was analyzed. Then the solution was placed in a vibration thermostat at 37° C. and an aliquot of 200 μl was taken in 0.5, 1 and 3 hours of incubation. 20 μl of a 10% solution of trichloroacetic acid was added to the selected sample and kept for 15 min at a temperature of minus 24° C., centrifuged at 6000 g for 5 min to precipitate the plasma proteins, supernatant was collected and its analysis was conducted.

The study of the stability of phenylcreatine compared to creatine in an aqueous solution and blood was carried out using reversed-phase HPLC using a chromatographic system Agilent 1220 Infinity LC System (USA).

Buffer A was 30% acetonitrile with 0.1% TFA

Buffer B was 70% acetonitrile with 0.1% TFA

The temperature of 50° C., detection 220 nm

Flow 1.5 ml/min.

Column XRbridge Peptide BEH C18 (“Waters”) 5 μm 300 Å 150*4.6 mm

The following gradient was used (Table 1).

TABLE 1
Gradient used for assessing phenylcreatine and creatine stability

| Time, min. | 0 | 20 | 21 |
|---|---|---|---|
| % A | 100 | 0 | 100 |
| % B | 0 | 100 | 0 |

To assess the stability of the analytes, the peak areas of the compounds were compared at the beginning of the experiment and at selected intervals (Table 2).

TABLE 2
Stability of phenylcreatine in comparison
with stability of creatine in blood serum

Stability in a blood serum

| Substance | 0 h | 0.5 h | 1 h | 3 h |
|---|---|---|---|---|
| phenylcreatine | 100% | 100% | 99% | 96% |
| creatine | 100% | 98% | 67% | 52% |

As follows from the data given, phenylcreatine has a high stability in the blood, and the concentration remained practically unchanged for 3 hours, while the creatine concentration in the human blood decreased to 52%.

### EXAMPLE 3. EVALUATION OF THE FUNCTIONAL STATE OF MICE IN A TREADMILL TEST WITH CREATINE AND PHENYLCREATINE

In order to find out whether phenylcreatine is a functional analogue of creatine, and also how much its effect is related to the strength of creatine, the functional state of the mice was assessed, namely, the body weight was measured, activity and endurance in the test on white mongrel mice were assessed—males weighing 18-22 g.

Two experimental groups of mice and one control group (10 mice in each group) were selected. Initially, the animals were of equal mass. The animals were kept in accordance with the rules adopted by the European Convention for the Protection of Vertebrates used for experimental and other purposes (European Convention for the Protection of Vertebrates used for Experiments or for Other Scientific Purposes (EST No. 123), Strasbourg, 18 March 1986, M., 1990, 12 pp.). Animals were kept in standard vivarium conditions. The animals were killed by decapitation in accordance with the “Rules for carrying out work using experimental animals”, approved by order of the Ministry of Health of the USSR No. 742 of 13 Nov. 1984 (Bolshakov O P, Neznanov N G, Babakhnyan R V Didactic and ethical aspects of research on biomodels and on laboratory animals//Qualitative clinical practice. 2002. No. 1. P.58-61).

Within 20 days, the animals received an aqueous solution of creatine in a dosage of 0.3 mg per gram of weight. The dosage is chosen according to the data that the daily intake of creatine in the amount of 20 g for adult men of average weight 75 kg for six days leads to an increase in the concentration of muscle creatine (Daniel Santarsieri TLS., Antidepressant efficacy and side-effect burden: a quick guide for clinicians Drugs in Context. 2015; 4: 1-12.). Upon administration, the drug was dissolved in 0.3 ml of water and injected into mice through a probe into the stomach daily in the morning, on an empty stomach. The animals of the control group received a similar volume of water. Phenylcreatine was also administered for 20 days in an amount of 50 mg per kg of body weight.

Weighing of animals was performed on the 1st, 5th, 10th, 15th and 20th days of the study, on an empty stomach, immediately before the administration of creatine, phenylcreatine or distilled water in the control.

Endurance of mice under physical exertion was assessed according to a standard procedure (Emirova L R Potention by citamins of the action of medicinal substances that increase the endurance of athletes: dis . . . medical doctor: 14.00.25. M., 2004. 125 pp.) for the duration of running in the treadmill test. The animals of each group were subjected to daily training loads in a high load power mode, which was modelled by running on a treadmill at a speed of 29-31 m/min. The duration of daily mice training was 5 minutes. Endurance of mice was tested on the 1st, 5th, 10th, 15th, 20th and 25th days of training against the background of administration of drugs (or distilled water in the control). Endurance testing was conducted under the same conditions as training. Endurance was tested 1 hour after drug administration (Petrenko E R Comparative pharmacological study of adaptogenic properties of ginseng preparations: dis . . . candidate of biological sciences: 14.00.25., St. Petersburg, 1998. 126 pp.) until fatigue, the criterion of which was the lack of reaction of mice to stimulation of the legs and tail by electric current (Stratienko E N Influence of phenylethyl substituted derivatives of 3-oxypridine on the physical working capacity of mice under conditions of hypobaric hypoxia: dis . . . . Medical Candidate of Sciences Bryansk, 1996. DSP. 201 pp.). Running time was recorded in seconds. The study was carried out at rest, an hour after the administration of creatine or phenylcreatine, and immediately after the end of the run in the treadmill.

Statistical processing of data was carried out in the program Statistica, for all data groups, using the Student's criterion.

The following results were obtained on the effect of administration of creatine and phenylcreatine on the body weight of mice.

Body weight of the animals of control (initially 19±2 g) and the experimental groups taking creatine (initially 18±2 g) and phenylcreatine (originally 18.6±2 g), changed insignificantly. There was a tendency to increase in mass in the experimental groups, weight gain was 9% for the group of animals that received creatine and 15.4% for the group of animals receiving phenylcreatine. The increase in the body weight of mice in the control group was 6.4%, the data are reliable at 95% significance level.

The following results were obtained concerning the effect of the intake of creatine and phenylcreatine on the endurance of mice. Dosages of 10 mg of phenylcreatine per animal and 300 mg of creatine per animal were used.

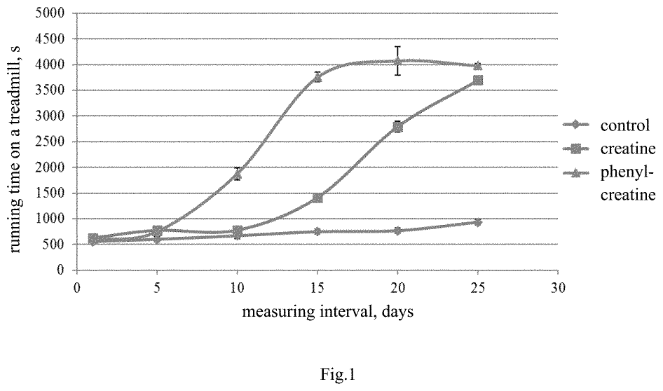

During the entire period of the study, the running time to total fatigue significantly increased on the 15th day of the study 2.8 times for animals from the experimental group receiving creatine and 6.4 times for the group receiving phenylcreatine, while in the control group the endurance increased in 1.1 times, on the 20th day of the study—4.5 times for animals from the experimental group that received creatine, and 6.9 times for the group receiving phenylcreatine, while in the animals of the control group, endurance increased by 1.4 times, on the 25th day of research—5.6 times for animals from the experimental group that received creatine, and 6.7 times for the group that received phenylcreatine, whereas in animals of the control group, endurance increased 1.7 times (Table 3, FIG. 1).

TABLE 3
Running time of mice (n = 15) at administration of creatine and phenylcreatine (M ± m)

| Groups | 1 d | 5 d | 10 d | 15 d | 20 d | 25 d |
|---|---|---|---|---|---|---|
| Controle, s | 556.71 ± 21.74 | 599.80 ± 43.00 | 669.00 ± 52.91 | 648.60 ± 46.52 | 789.50 ± 56.40 | 934.6 ± 76.3 |
| The group receiving | 626.00 ± 32.25 | 772.60 ± 33.07 | 775.80 ± 37.01 | 1811.30 ± 85.82 | 2794.70 ± 103.22 | 3697.5 ± 1373 |
| creatine, s | | | | | | |
| The group receiving | 588.23 ± 25 | 747.23 ± 17.06 | 1874.12 ± 114.43 | 3756.77 ± 97.02 | 4076.45 ± 276.57 | 3981.4 ± 202.8 |
| phenylcreatine, s | | | | | | |
| P, groups receiving | P > 0.05 | P ≤ 0.05 | P > 0.05 | P ≤ 0.05 | P ≤ 0.05 | P ≤ 0.05 |
| creatine and | | | | | | |
| phenylcreatine | | | | | | |

As a result of the course receiving, both creatine and phenylcreatine, a significant increase in endurance is observed for 20 days, starting from the 15th day of intake for creatine and from the 10th day of intake for phenylcreatine, and increases until it is completed. The maximum effect from the intake of phenylcreatine is already on the 15th day, that is, it increases 2 times faster than in the experimental group receiving creatine. The results obtained allow us to conclude that the intake of creatine contributes to an increase in endurance and ability to work. Taking of phenylcreatine further enhances this effect, and also promotes the body to the peak of physical abilities in preparation for physical exertion. This effect of phenylcreatine persisted even in the absence of sleep.

### EXAMPLE 4. EFFECTIVENESS OF PHENYLCREATINE IN THE THERAPY OF EXTRASYSTOLES

One of the most important factors of arrhythmogenesis and the appearance of extrasystoles is the activation of the sympathoadrenal system. This circumstance determined the necessity of investigating phenylcreatine, proposed by the author of the present invention, on the model of adrenal arrhythmia (extrasystole) in rats (Kushakovsky M S, Heart arrhythmias: a guide for physicians, St. Petersburg, Hippocrates 1992).

In a control series of experiments, in all animals, 12 seconds after injection of epinephrine hydrochloride at a dose of 50 mg/kg, polytopic ventricular extrasystole occurred in all animals. The number of ventricular extrasystoles before transition to tachycardia averaged 32±6. The duration of such arrhythmias was 80±21 seconds. In 50% of cases, it passed into the ventricular tachycardia. The duration of tachycardia was, on average, 86±12 seconds.

With the introduction of phenylcreatine, proposed by the present inventor in an amount of 20 mg per animal, half an hour before adrenaline hydrochloride, the number of ventricular extrasystoles was 12±4. The duration of the arrhythmia was 60±14 seconds. There was no transition to tachycardia.

Additionally, the following study was carried out. Man, 36 years old, professional sportsman (15 years of experience in power triathlon). Supraventricular extrasystoles with a frequency of 4 times in 24 hours were observed according to holter monitoring. Extrasystoles were very poorly tolerated, there were complaints of discomfort and a decrease in the quality of life (neurosis-like condition). He took phenylcreatine in an amount of 2 mg per 1 kg per day, for 14 days. A gradual decrease in the amount and strength of extrasystoles since the initiation of phenylcreatine was noted, after the course extrasystoles completely disappeared. Within 3 months after the course the holter monitoring does not fix supraventricular extrasystoles.

### EXAMPLE 5. EVALUATION OF NOOTROPIC ACTION OF PHENYLCREATINE

The experiments were performed on male Wistar rats born in September 2014 (experiments were conducted in November 2016). The animals were kept in standard plastic cells at an air temperature of 21-23° C. They received a balanced granular food and drinking water without restrictions. The work was carried out in compliance with the principles of the Helsinki Declaration on Humane Treatment of Animals.

Rats were divided into 2 groups. The rats of the first test group received 10 mg of phenylcreatine per animal daily for a month with drinking water. The rats of the second test group received water. As a control in the experiment, rats born in May 2016 (the third group, young rats) were used.

In the experiment, a shuttle maze was used to evaluate neuropsychiatric processes, primarily cognitive processes (Navakatikyan M A, Platonov L L, 1988). At the end of the labyrinth there was a food reinforcement (a piece of cheese with a mass of 200 mg).

The time of the experiment to find the exit from the labyrinth was 5 minutes. During the experiment, the time of passing the labyrinth, the number of rats reaching the end of the labyrinth, the number of vertical racks were recorded.

If consider age dynamics in terms of locomotor and cognitive activity, it decreases with age 5 times (from 3 months to 24 months) (Anisimov V N, 2001).

Results:

Group 1. (Old rats 25 months plus phenylcreatine)

The number of racks per minute—1.2±0.44

The number of rats reaching the end of the labyrinth in 5 minutes—50%

The transit time of the labyrinth is 2±0.22 minutes

Group 2. (old rats 25 months)

Number of racks per minute—2±0.56

The number of rats reaching the end of the labyrinth in 5 minutes—10%

The transit time of the labyrinth is 5±0.42 minutes

Group 3. (young rats 6 months)

Number of racks per minute—0.3±0.21

The number of rats reaching the end of the labyrinth in 5 minutes—70%

Time of passage of the labyrinth is 1±0.12 minutes

The results obtained confirm the possibility of using phenylcreatine according to the invention as a nootropic agent.

It should also be noted that, with all the studies conducted, the negative effects of phenylcreatine according to the invention were not detected, which indicates its safety.

1. Phenylcreatine of formula

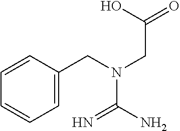 [SMILES: N=C(N)N(CC(=O)O)Cc1ccccc1]

2. The use of phenylcreatine according to claim 1 as a functional analogue of creatine.

3. The use of phenylcreatine according to claim 1 for the prevention or treatment of arrhythmia.

4. The use of phenylcreatine according to claim 1 as a nootropic agent.

5. A method of producing phenylcreatine according to claim 1, comprising mixing cyanamide, pre-exposed to ammonia in catalytic amounts, with N-benzylglycine, and exposure for 24-96 hours at a temperature from +20° C. to +65° C.
